(19) United States
(12) Patent Application Publication
(10) Pub. No.: US 2014/0314819 A1
(43) Pub. Date: Oct. 23, 2014

(54) COMPOSITIONS AND METHODS FOR TREATING DENTAL CONDITIONS

(71) Applicants: Dale S. Scherl, Lawrence, KS (US); Dennis Jewell, Lawrence, KS (US); Philip W. Toll, Valley Falls, KS (US); Kevin A. Hahn, Topeka, KS (US)

(72) Inventors: Dale S. Scherl, Lawrence, KS (US); Dennis Jewell, Lawrence, KS (US); Philip W. Toll, Valley Falls, KS (US); Kevin A. Hahn, Topeka, KS (US)

(73) Assignee: HILL'S PET NUTRITION, INC., Topeka, KS (US)

(21) Appl. No.: 14/362,925

(22) PCT Filed: Aug. 30, 2012
(86) PCT No.: PCT/US2012/053038

§ 371 Date: Jun. 5, 2014

Related U.S. Application Data
(60) Provisional application No. 61/570,121, filed on Dec. 13, 2011.

(51) Int. Cl. A61K 8/365; A61Q 11/00; A61K 8/02

(52) U.S. Cl. CPC A61K 8/365; A61K 8/0233; A61Q 11/00
USPC 424/401; 424/55; 424/54; 424/50

(57) ABSTRACT

The present disclosure provides methods and compositions for treating or reducing a dental condition in an animal, particularly in canine and feline companion animals. Compositions useful in the disclosed methods comprise an effective amount of lactic acid. The disclosed compositions may also contain oral health promoting effective amounts of at least one antioxidant. Dental conditions treated or reduced according to the disclosed methods include dental plaque, calculus, tooth staining, halitosis, stomatitis, gingivitis, periodontitis, and combinations thereof.

### CROSS REFERENCE TO RELATED APPLICATIONS

This application claims priority to U.S. Provisional Application No. 61/570,121, filed on 13 Dec. 2011, which is incorporated herein by reference.

### FIELD OF THE INVENTION

The present invention relates to methods and compositions useful for treatment or reduction of dental conditions in an animal, particularly in a companion animal.

### BACKGROUND OF THE INVENTION

Virtually all domestic companion animals, e.g., canine and feline companion animals, as well as many other animals, accumulate dental plaque and calculus (tartar). These accumulations can lead to the animal's affliction with gingivitis, which typically progresses into periodontitis that, in turn, leads to serious systemic diseases and conditions. Animals afflicted with these dental conditions are also generally afflicted with halitosis and stomatitis.

Dental plaque is formed as a result of the bacterial colonization of teeth. Dental plaque is a layer of bacteria on the erupted surfaces of teeth and in the gingival crevice. More specifically, dental plaque is a biofilm, usually a pale yellow that develops naturally on the teeth. Like any biofilm, dental plaque is formed by colonizing bacteria trying to attach themselves to a smooth surface, e.g., a tooth. However, the presence of plaque comprising specific bacterial species can lead to the development of dental caries and periodontal disease.

Dental calculus, or tartar, is a recurring calcified deposit on the surfaces of the teeth of many animals, including domesticated dogs and cats, humans, and primates. It is generally recognized that dental calculus develops in a sequential process that involves the accumulation of dental plaque and the subsequent calcification of the plaque by saliva, which contains very high concentrations of calcium and phosphate.

Thus, calculus or tartar is a form of hardened dental plaque, caused by the continual accumulation of minerals from saliva or plaque on the teeth. The rough surface of tartar provides an ideal medium for further plaque formation, threatening the health of the gingiva.

Brushing and flossing can remove plaque from which calculus forms; however, once formed, it is too hard and firmly attached to be removed with a toothbrush. The accumulation of plaque and its sequential solidification or calcification as dental calculus or tartar eventually causes the gingiva to become irritated and inflamed, i.e., the condition referred to as gingivitis. Gingivitis in turn can progress and develop into periodontitis, which is characterized by a loss of the connective tissue fibers that attach the gums to the teeth and bone that surrounds the tooth. Calculus (tartar) is detrimental to gingival health because it serves as a substrate for increased plaque formation and retention.

Once formed, calculus deposits can only be removed through dental prophylaxis or other mechanical procedures. Thus, the treatment of dental tartar is of importance not only for cosmetic reasons, but also because its role in the development of gingivitis, stomatitis, periodontitis, and the resultant systemic infections, alveolar bone recession, interference in the normal mastication process, tooth loss and adverse mouth and breath odors.

Since the microorganisms comprising dental plaque are recognized as the etiological agents responsible for the development of tartar and the subsequent inflammation of the oral soft tissues (e.g., gingivitis), it is well accepted that measures that remove tartar or inhibit or prevent formation of dental tartar will reduce gingivitis, and thereby obviate or inhibit the progression from tartar accumulation to gingivitis, and ultimately to periodontitis.

It is also apparent that compositions or methods that might be available for treatment of dental conditions in humans are not directly applicable for use in animals in light of, inter alia, the differences in compliance, cooperation, and temperament between animals, including companion canine and feline animals, and humans. Accordingly, a need exists for methods that can effectively control, reduce, or inhibit formation of dental plaque and calculus in animals. Additionally, a need exists for food products, compositions, solutions or methods that are useful for the effective control, reduction, or inhibition of formation of dental plaque and calculus in animals.

### BRIEF SUMMARY OF THE INVENTION

Provided herein are methods for treating or reducing a dental condition in an animal comprising feeding an effective amount of a composition comprising at least 0.8% lactic acid to an animal in need of such treatment or reduction. In certain embodiments, the dental condition is selected from the group consisting of dental plaque, calculus, tooth staining, halitosis, stomatitis, gingivitis, periodontitis and combinations thereof.

In certain embodiments, the method for treating or reducing a dental condition in an animal comprises feeding the animal in need thereof, a composition comprising from about 0.8% to about 5% lactic acid. In other aspects, the composition comprises about 1.0 to about 1.2% lactic acid. In still a further aspect, the composition comprises about 1.1 to about 1.5% lactic acid.

In other embodiments, the method for treating or reducing a dental condition in an animal comprises feeding the animal in need thereof, a composition that comprises at least 0.8% lactic acid, from about 0.8% to about 5% lactic acid, from about 1.0% to about 1.9%, from about 1.1% to about 1.8% lactic acid, about 1.0% to about 1.2% lactic acid, or about 1.1% to about 1.5% lactic acid, and further comprises an antioxidant.

In certain embodiments, the animal in need of treatment or reduction is a companion animal. In one aspect, the animal is a canine while in another, the animal is a feline.

In one embodiment the composition comprising at least 0.8%, from about 0.8% to about 5% lactic acid, about 1.0% to about 1.2%, or about 1.1% to about 1.5% lactic acid, is a companion animal pet food composition that, in one aspect, is a nutritionally-complete companion animal pet food composition. In still other embodiments, the disclosed method for treating or reducing a dental condition in an animal comprises feeding the animal in need thereof, a composition comprising at least 0.8%, at least 0.8% to 5%, about 1.0% to about 1.2%, about 1.1% to about 1.5% of lactic acid, wherein the composition is in the form of a treat, a dietary supplement, or a coating applied to a chew or applied to an edible toy.

Also provided herein are pet food compositions that are suitable for treating or reducing a dental condition in an animal, e.g., pet food compositions that are suitable for use in the methods disclosed herein. In certain aspects of this embodiment, the dental condition is selected from the group consisting of dental plaque, calculus, tooth staining, halitosis, stomatitis, gingivitis, periodontitis and combinations thereof. In other aspects of this embodiment, the animal in need of treatment or reduction in a dental condition is a companion animal. In one aspect, the animal is a canine while in another, the animal is a feline.

In one embodiment, the pet food compositions of the present disclosure are nutritionally complete companion animal pet food compositions. In one aspect of this embodiment, the pet food composition comprises at least 0.8% lactic acid, from about 10% to about 50% protein, from about 4% to about 25% fat, from about 20% to about 70% carbohydrate, and from about 4% to about 20% crude fiber.

In another specific embodiment, a pet food composition of the present disclosure also comprises an antioxidant, which, for example, can be antioxidant is selected from the group consisting of vitamin C, vitamin E, vitamin A, lipoic acid, astaxanthin, beta-carotene, L-carnitine, coenzyme Q10, glutathione, lycopene, lutein, N-acetylcysteine, soy isoflavones, S-adenosylmethionine, taurine, tocotrienols, spinach, tomato, citrus fruit, grape, carrot, broccoli, green tea, ginkgo biloba, corn gluten meal, rice bran, algae, curcumin, marine oil, fruits, vegetables, yeast, carotenoids, flavonoids, polyphenols, and mixtures thereof.

The compositions can be fed to the animal over a period of at least one month, at least two months, at least three months, at least six months or at least twelve months.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION OF THE INVENTION

The following description of the preferred embodiment(s) is merely exemplary in nature and is in no way intended to limit the invention, its application, or uses.

As used herein and in the appended claims, the singular forms “a,” “an,” and “the” include plural references unless the context clearly dictates otherwise.

The term “animal” as used herein refers to non-human animals, such as mammals and is particularly applicable to companion animals. Non-human mammals include non-human primates such as monkeys, chimpanzees, etc. Farm animals include goats, sheep, swine, cattle, etc. Wild and zoo animals include wolves, bears, deer, giraffes, elephants, etc. Non-mammalian animals include birds and working animals include horses.

The term “companion animal” used in the present invention includes any non-human animal suitable for being kept as a pet by humans including a dog, a cat, and a rodent. The compositions of the invention are in certain embodiments for the treatment of cats and/or dogs.

The term “dog” includes those dogs which are companion animals such as Canis familialis, working dogs and the like. The term dog is synonymous with the term canine.

The term “cat” includes those cat, which are companion animals known as domestic cats or house cats. The term cat is synonymous with the term feline.

As used herein, “an amount effective,” “an effective amount,” and like terms refer to that amount of a compound, material or composition as described herein that may be effective to achieve a particular biological result, e.g., to treat or reduce a dental condition selected from the group consisting of dental plaque, calculus, tooth staining, gingivitis, periodontitis, stomatitis, halitosis, and combinations thereof. Such results may be achieved, for example, by administration of the compositions of the present invention to an animal. An effective amount may be based on several factors, including the particular animal's sex, age, and weight.

In one embodiment, the method methods disclosed herein are effective to reduce accumulation of dental plaque or calculus. Accordingly, in other embodiments the method is effective to promote gingival and periodontal health as well.

The present disclosure relates to any animal, e.g., to a mammal and more specifically to a companion animal that may benefit from feeding the formulations disclosed herein. The term “companion animal” refers to any animal that lives in close association with humans and includes, but is not limited to, canines and felines. For example, it is contemplated herein that this term may also encompass any animal whose diet may be controlled by humans and which may benefit from feeding the formulations disclosed herein. These animals may include, e.g., domesticated farm animals (e.g. cattle, horses, swine, etc.) as well as undomesticated animals held in captivity, e.g. in zoological parks and the like. In a specific embodiment, the animal is a feline or canine.

The present invention may be suitable for use with animals in various stages in life, including lactation, weaning, growth, adult, senior, and geriatric. In specific embodiments, the animal is an adult, senior, or geriatric animal, and particularly, an adult animal.

The term “treating,” in its various grammatical forms in relation to the present disclosure, refers to preventing, curing, reversing, attenuating, alleviating, ameliorating, minimizing, suppressing or halting the deleterious effects of one or more of the dental conditions disclosed herein.

Unless otherwise specified, all percentages and amounts expressed herein and elsewhere in the specification should be understood to refer to percentages by weight. The amounts given are based on the active weight of the material. Except to the extent stated otherwise, all percentages used in this specification are weight percentages on a dry matter basis. The phrase “dry matter basis” means the component concentration in the composition after any moisture in the composition is removed.

Issues related to oral health are some of the most common diagnoses made by small animal veterinarians in the United States; in fact, dental calculus and gingivitis are the two most common diagnoses in canines and feline. A common etiological feature of calculus and gingivitis is bacterial plaque which if left to accumulate and solidify can lead to periodontal disease progression resulting in tissue destruction, loss of functionality, tooth loss, and the potential for systemic infection that can ultimately affect overall health. The methods and compositions disclosed herein interfere with early steps in the underlying, progressive cascade of events and factors leading to dental conditions that include dental plaque, calculus, tooth staining, halitosis, stomatitis, gingivitis, periodontitis.

Applicants have discovered that feeding canines compositions of the present disclosure comprising a high level of lactic acid inhibited both formation of dental plaque, calculus and dental stains. In particular, it has been discovered that feeding canines a pet food composition comprising about 1.1% to about 1.5% lactic acid and feeding felines a composition comprising about 1.0% to about 1.2% lactic acid inhibited formation of dental plaque, calculus and dental stains. Accordingly, in view of the above and the progressive nature of the identified dental conditions, the disclosed methods for treatment or reduction of tartar formation and tooth staining are also applicable to treatment and reduction of halitosis, stomatitis, gingivitis, periodontitis, and combinations thereof.

In one embodiment, the methods of the disclosure are directed to treating or reducing a dental condition in an animal in need of such treatment or reduction, comprising feeding that animal a composition comprising at least 0.8% lactic acid. In other aspects of this embodiment, the composition comprises from about 0.8% to about 5% lactic acid. In further aspects of this embodiment, the composition comprises from about 1.0% to about 1.9%, from about 1.1% to about 1.8% lactic acid, from about 1.2% to about 1.7% lactic acid; or about 0.8%, about 0.9%, about 1.0%, about 1.1%, about 1.2%, about 1.3%, about 1.4%, about 1.5%, about 1.6%, about 1.7%, 1.8%, 1.9%, or about 2.0% lactic acid. In a specific embodiment, the composition comprises about 1.0% to about 1.2% lactic acid. In another specific embodiment, the composition comprises about 1.1% to about 1.5% lactic acid.

In certain embodiments, the dental condition to be treated or reduced is selected from the group consisting of dental plaque, calculus, tooth staining, halitosis, stomatitis, gingivitis, periodontitis and combinations thereof. In a particular aspect, the dental condition to be treated or reduced is dental plaque. In another aspect, the dental condition to be treated or reduced is calculus. In another aspect, the dental condition to be treated or reduced is tooth staining. In a further aspect, the dental condition to be treated or reduced is halitosis. In a still further aspect, the dental condition to be treated or reduced is stomatitis. In one certain aspect, the dental condition to be treated or reduced is gingivitis, while in another certain aspect, the dental condition to be treated or reduced is periodontitis.

The methods of this invention contemplate administration of any of a variety of compositions comprising lactic acid to the animal. Contemplated compositions suitable for ingestion by a companion animal include, for example, foods, supplements, treats, snacks and toys (typically chewable and consumable toys).

In one embodiment, the composition comprising lactic acid can be fed to the animal as a component of its food intake. The food intake of the animal can meet its ordinary nutritional requirements, which a skilled artisan can determine based upon the animal's species, age, sex, weight, and other factors. For example, a typical diet for a canine of 1-6 years of age contains on a dry matter basis about 23% protein, about 15% fat, about 0.6% phosphorus, 0.6% calcium and about 0.3% sodium; and, for older (“mature”) canines and felines, a typical diet can be, for example, as provided in Table 1.

TABLE 1
Typical Composition Of Diet For Mature Canines And Felines

| Component | | |
|---|---|---|
| (% Dry Matter) | Canine | Feline |
| Crude protein | 15-25 | 25-50 |
| Crude Fat | 7-20 | 10-30 |
| Crude Fiber | >2 | <10 |
| Carbohydrate | 20-70 | 10-50 |
| Calcium | 0.5-1.2 | 0.6-1.5 |
| Phosphorous | 0.25-1.2 | 0.5-1.5 |
| Sodium | 0.15-0.5 | 0.15-0.5 |
| Magnesium | 0.05-0.2 | 0.05-0.15 |
| Energy density¹ | 3.0-4.5 | 3.0-5.0 |

¹kCal ME (metabolizable energy) per kg food (dry matter)

In specific embodiments, the methods of the disclosure are directed to treating or reducing a dental condition in an animal in need of such treatment or reduction, comprising feeding that animal a composition comprising at least 0.8% lactic acid, from about 0.8% to about 5% lactic acid, about 1.0% to about 1.2% or about 1.1% to about 1.5% lactic acid, wherein the composition is a companion animal pet food composition. In certain aspects of this embodiment, the compositions fed to the animal in need thereof comprise from about 1.0% to about 1.9%, from about 1.1% to about 1.8% lactic acid, from about 1.2% to about 1.7% lactic acid; or about 0.8%, about 0.9%, about 1.0%, about 1.1%, about 1.2%, about 1.3%, about 1.4%, about 1.5%, about 1.6%, about 1.7%, 1.8%, 1.9%, or about 2.0% lactic acid. In a specific embodiment, the composition comprises about 1.0% to about 1.2% lactic acid. In another specific embodiment, the composition comprises about 1.1% to about 1.5% lactic acid. In one aspect of such embodiments, the companion animal pet food composition comprises suitable sources of protein, carbohydrate, fat, and fiber.

In a particular aspect of this embodiment, the methods of the disclosure comprise feeding a composition of the disclosure, which composition is a companion animal pet food composition comprising from about 10% to about 50%, from about 15% to about 40%, and from about 20% to about 30% protein. In a specific aspect, the methods of the disclosure comprise feeding a composition of the disclosure, which composition is a companion animal pet food composition comprising about 25% protein.

In a particular aspect of this embodiment, the methods of the disclosure comprise feeding a composition of the disclosure, which composition is a companion animal pet food composition comprising from about 30% to about 70%, from about 35% to about 65%, and from about 40% to about 60% carbohydrate. In a specific aspect, the methods of the disclosure comprise feeding a composition of the disclosure, which composition is a companion animal pet food composition comprising about 50% carbohydrate.

In still another aspect of this embodiment, the methods of the disclosure comprise feeding a composition of the disclosure, which composition is a companion animal pet food composition comprising from about 4% to about 20%, from about 5% to about 16%, from about 6% to about 14%, and from about 8% to about 12% fat. In a specific aspect, the methods of the disclosure comprise feeding a composition of the disclosure, which composition is a companion animal pet food composition comprising about 10% fat.

In a another aspect of this embodiment, the methods of the disclosure comprise feeding a composition of the disclosure, which composition is a companion animal pet food composition comprising from about 4% to about 20%, from about 6% to about 18%, from about 8% to about 16% protein, and from about 10% to about 14% crude fiber. In a specific aspect, the methods of the disclosure comprise feeding a composition of the disclosure, which composition is a companion animal pet food composition comprising about 12% crude fiber.

In a particular aspect of this embodiment, the methods of the disclosure comprise feeding a composition of the disclosure, which composition is a companion animal pet food composition comprising about 33% protein, about 30% carbohydrate, about 20% fat, about 8% crude fiber and about 1.0% to about 1.2% lactic acid.

In another particular aspect of this embodiment, the methods of the disclosure comprise feeding a composition of the disclosure, which composition is a companion animal pet food composition comprising about 25% protein, about 50% carbohydrate, about 10% fat, about 12% crude fiber and about 1.1% to about 1.5% lactic acid.

The compositions can be fed to the animal over a period of at least one month, at least two months, at least three months, at least six months or at least twelve months.

The pet food compositions disclosed above are particularly suitable for feeding to canines in need of dental treatment. In other embodiments, pet food compositions particularly suitable for use in the disclosed methods of treatment of feline companion animals generally have some higher levels of protein but lower levels of carbohydrate and fiber than described above.

Therefore, in another embodiment, the methods of the disclosure are directed to treating or reducing a dental condition in an animal in need of such treatment or reduction, comprising feeding that animal, which can be a feline companion animal, a composition comprising at least 0.8% lactic acid, from about 0.8% to about 5% lactic acid, about 1.0% to about 1.2% or about 1.1% to about 1.5% lactic acid, wherein the composition is a companion animal pet food composition. In certain aspects of this embodiment, the compositions fed to the animal in need thereof comprise from about 1.0% to about 1.9%, from about 1.1% to about 1.8% lactic acid, from about 1.2% to about 1.7% lactic acid; or about 1.0%, about 1.1%, about 1.2%, about 1.3%, about 1.4%, about 1.5%, about 1.6%, about 1.7%, 1.8%, 1.9%, or about 2.0% lactic acid. In one specific embodiment, the composition comprises about 1.0% to about 1.2% lactic acid. In another specific embodiment, the composition comprises about 1.1% to about 1.5% lactic acid. In one aspect of such embodiments, the companion animal pet food composition comprises suitable sources of protein, carbohydrate, fat, and fiber.

In a particular aspect of this embodiment, the methods of the disclosure comprise feeding a composition of the disclosure, which composition is a companion animal pet food composition comprising from about 20% to about 45%, from about 25% to about 40%, and from about 30% to about 35% protein. In a specific aspect, the methods of the disclosure comprise feeding a composition of the disclosure, which composition is a companion animal pet food composition comprising about 33% protein.

In a particular aspect of this embodiment, the methods of the disclosure comprise feeding a composition of the disclosure, which composition is a companion animal pet food composition comprising from about 15% to about 45%, from about 20% to about 40%, and from about 25% to about 35% carbohydrate. In a specific aspect, the methods of the disclosure comprise feeding a composition of the disclosure, which composition is a companion animal pet food composition comprising about 30% carbohydrate.

In still another aspect of this embodiment, the methods of the disclosure comprise feeding a composition of the disclosure, which composition is a companion animal pet food composition comprising from about 5% to about 35%, from about 10% to about 30%, and from about 15% to about 25% fat. In a specific aspect, the methods of the disclosure comprise feeding a composition of the disclosure, which composition is a companion animal pet food composition comprising about 20% fat.

In a another aspect of this embodiment, the methods of the disclosure comprise feeding a composition of the disclosure, which composition is a companion animal pet food composition comprising from about 4% to about 15%, from about 5% to about 12%, from about 6% to about 10% protein, and from about 7% to about 9% crude fiber. In a specific aspect, the methods of the disclosure comprise feeding a composition of the disclosure, which composition is a companion animal pet food composition comprising about 8% crude fiber.

In a particular aspect of this embodiment, the methods of the disclosure comprise feeding a composition of the disclosure, which composition is a companion animal pet food composition comprising about 33% protein, about 30% carbohydrate, about 20% fat, about 8% crude fiber and about 1.0% to about 1.2% lactic acid.

In one illustrative example, a nutritionally-complete companion animal pet food composition for canine companion animals may further comprise, inter alia, vitamins, minerals and other additives. In one aspect of this embodiment, this pet food composition comprises at least 0.8% lactic acid, about 0.8% to about 5% lactic acid, from about 1.0% to about 1.9%, from about 1.1% to about 1.8% lactic acid, from about 1.2% to about 1.7% lactic acid; or about 0.9%, about 1.0%, about 1.1%, about 1.2%, about 1.3%, about 1.4%, about 1.5%, about 1.6%, about 1.7%, 1.8%, 1.9%, or about 2.0% lactic acid, about 1.0% to about 1.2% or about 1.1% to about 1.5% lactic acid, and may further comprise whole grain corn, soybean mill run, chicken by-product meal, powdered cellulose, corn gluten meal, soybean meal, chicken liver flavor, soybean oil, flaxseed, caramel color, iodized salt, L-lysine, choline chloride, potassium chloride, vitamin E supplement, vitamins (L-ascorbyl-2-polyphosphate (source of vitamin C), vitamin E supplement, niacin, thiamine mononitrate, vitamin A supplement, calcium pantothenate, biotin, vitamin B₁₂ supplement, pyridoxine hydrochloride, riboflavin, folic acid, vitamin D₃ supplement), minerals (ferrous sulfate, zinc oxide, copper sulfate, manganous oxide, calcium iodate, sodium selenite), taurine, L-carnitine, preserved with mixed tocopherols and citric acid, phosphoric acid, beta-carotene, and rosemary extract.

In another illustrative example, a nutritionally complete companion animal pet food composition for feline companion animals may further comprise, inter alia, vitamins, minerals and other additives. In one aspect of this embodiment, this pet food composition comprises at least 0.8% lactic acid, about 0.8% to about 5% lactic acid, about 1.0% to about 1.2% or about 1.1% to about 1.5% lactic acid and may further comprise brewers rice, corn gluten meal, chicken by-product meal, powdered cellulose, whole grain corn, chicken liver flavor, animal fat (preserved with mixed tocopherols and citric acid), soybean mill run, calcium sulfate, choline chloride, potassium chloride, iodized salt, taurine, DL-methionine, vitamin E supplement, vitamins (L-ascorbyl-2-polyphosphate (source of vitamin C), vitamin E supplement, niacin, thiamine mononitrate, vitamin A supplement, calcium pantothenate, riboflavin, biotin, vitamin B₁₂ supplement, pyridoxine hydrochloride, folic acid, vitamin D₃ Supplement), minerals (ferrous sulfate, zinc oxide, copper sulfate, manganous oxide, calcium iodate, sodium selenite), L-carnitine, preserved with mixed tocopherols and citric acid, phosphoric acid, beta-carotene, and rosemary extract.

In another embodiment, the methods of the disclosure comprise feeding a composition of the disclosure, which composition is a companion animal pet food composition as described above, and further comprising an anti-oxidant. In particular, such compositions comprise an oral health-promoting effective total amount of at least one antioxidant. In certain aspects, the antioxidant is selected from the group consisting of vitamin C, vitamin E, vitamin A, lipoic acid, astaxanthin, beta-carotene, L-carnitine, coenzyme Q10, glutathione, lycopene, lutein, N-acetylcysteine, soy isoflavones, S-adenosylmethionine, taurine, tocotrienols, spinach, tomato, citrus fruit, grape, carrot, broccoli, green tea, ginkgo biloba, corn gluten meal, rice bran, algae, curcumin, marine oil, fruits, vegetables, yeast, carotenoids, flavonoids, polyphenols, and mixtures thereof.

In certain embodiments, the antioxidant-comprising composition can comprise vitamin E, vitamin C, or both vitamin E and vitamin C. In one aspect of this embodiment, the vitamin E content of a composition can be at least about 100 ppm, illustratively about 100 to about 5000 ppm, about 250 to about 2500 ppm, or about 500 to about 1500 ppm. In another aspect of this embodiment, the vitamin C content of a composition can be at least about 10 ppm, illustratively about 10 ppm to about 10,000 ppm, or about 20 to about 2000 ppm, or about 25 to about 500 ppm.

In certain embodiment, compositions of the present invention are formulated as companion animal pet food compositions. In one aspect of this embodiment, the pet food compositions are nutritionally complete pet food compositions.

In particular embodiments, the pet food compositions, and particularly the nutritionally-complete pet food compositions of the present disclosure, can be prepared in a dry form using conventional processes. In one contemplated embodiment, dry ingredients, including, for example, animal protein sources, plant protein sources, grains, etc., are ground and mixed together. Moist or liquid ingredients, including fats, oils, animal protein sources, water, etc., are then added to and mixed with the dry mix. The mixture is then processed into kibbles or similar dry pieces. Kibble is often formed using an extrusion process in which the mixture of dry and wet ingredients is subjected to mechanical work at a high pressure and temperature, and forced through small openings and cut off into kibble by a rotating knife. The wet kibble is then dried and optionally coated with one or more topical coatings which can include, for example, flavors, fats, oils, powders, and the like. Kibble also can be made from the dough using a baking process, rather than extrusion, wherein the dough is placed into a mold before dry-heat processing. Kibble also can be made from a food matrix undergoing pelletization. It particular aspects of this embodiment, lactic acid is incorporated into the food composition by adding it to the above-described mixtures before extrusion or by coating the extruded kibble or pellets with, for example, lactic acid as an ingredient of a topical coating.

In another aspect of this embodiment, lactic acid is also incorporated into the food composition by adding it to the above-described mixtures before extrusion or by coating the extruded kibble or pellets with, for example, the lactic acid as an ingredient of a topical coating.

Treats of the present invention can be prepared by, for example, an extrusion or baking process similar to those described above for dry food. Other processes also can be used to either apply a coating comprising lactic acid on the exterior of existing treat forms, or injecting lactic acid into an existing treat form. In a similar manner, treats of the present disclosure may also comprise one or more oxidants that may also be included in a coating comprising the exterior of existing treat forms, or injected into an existing treat form.

In another embodiment, the composition is a food supplement comprising lactic acid. Supplements include, for example, a feed or pet food used with another feed or pet food to improve the nutritive balance or performance of the total. Contemplated supplements include compositions that are fed undiluted as a supplement to other feeds or pet foods, offered free choice with other parts of an animal's ration that are separately available, or diluted and mixed with an animal's regular feed or pet food to produce a complete feed or pet food. Supplements can be in various forms including, for example, powders, liquids, syrups, pills, encapsulated compositions, etc.

In another embodiment, the composition is a treat comprising lactic acid. Treats include, for example, compositions that are given to an animal to entice the animal to eat during a non-meal time. Treats can be nutritional, wherein the composition comprises one or more nutrients, and can, for example, have a composition as described above for food. Non-nutritional treats encompass any other treats that are non-toxic. A lactic acid containing composition can, for example, be coated onto the treat, incorporated into the treat, or both.

In another embodiment, the composition is a toy comprising lactic acid. Toys include, for example, chewable toys. Contemplated toys for dogs include, for example, artificial bones. The lactic acid or lactic acid containing composition, for example, can be present in a coating on the surface of the toy or on the surface of a component of the toy, or can be incorporated partially or fully throughout the toy, or both. In a contemplated embodiment, lactic acid is orally accessible by the intended user.

It should be recognized that this disclosure contemplates both partially consumable toys (e.g., toys comprising plastic components) and fully consumable toys (e.g., rawhides and various artificial bones). It should be further recognized that this invention contemplates toys for both human and non-human use, particularly for companion, farm, and zoo animal use, and particularly for canine or feline use.

The terms “treat” and “toy” can be considered interchangeable for the purposes of this specification. However, in general a treat is fully edible and a toy in accordance with the invention has an edible coating.

In preparing a composition of the present invention, lactic acid can, for example, be incorporated into the composition during formulation processing, such as during and after mixing of other components of the composition. Distribution of these components into the composition can be accomplished by any conventional method including standard mixing procedures.

In certain embodiments, the compositions of the present disclosure, whether a companion animal pet food composition, dietary supplement, treat or toy, may further comprise at least one of a commercial liquid palatant enhancer or other flavor composition to create a novel flavor palatant which can then be included within or topically applied to the composition. Suitable commercial liquid palatant enhancers for use with compositions of the present disclosure can include any known or commercially available liquid palatant enhancers commercially available from pet food palatant enhancer or other flavor suppliers known to those of skill in the art. In one illustrative aspect, the kibble, treat, or toy can be coated with a composition comprising lactic acid and a carrier, wherein the carrier comprises protease-treated minced animal by-product, amino acids, one or more reducing sugars and thiamin.

The invention is not limited to the particular methodology, protocols, and reagents described herein because they may vary. Further, the terminology used herein is for the purpose of describing particular embodiments only and is not intended to limit the scope of the present invention.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

### EXAMPLES

### Example 1

### Lactic Acid Inhibition of Dental Plaque, Calculus, and Staining in Felines (Cats): Twelve Month Feeding Study

This study was designed to assess the effect of lactic acid on oral substrate accumulation (dental plaque, calculus, and tooth stain) in cats. Initially, all cats received a professional dental prophylaxis at this baseline using standard, well-established procedures. Six and twelve months later, dental substrate accumulation (dental plaque, calculus, and tooth stain) was quantified using the Logan-Boyce quantification method (See Logan E I, Boyce. Oral health assessment in dogs: parameters and methods. J. Vet Dent; 11(2):58-63.)

Plaque is measured by applying a disclosing solution (2% aqueous eosin) to the animal's teeth. The teeth to be scored are divided into gingival and occlusal halves, and each half is assigned a separate numerical score for plaque coverage (percentage of the tooth area stained) and thickness (intensity of staining) The score for each tooth half is calculated by multiplying the coverage and thickness scores. The gingival and occlusal scores are then added together for a tooth score. The sum of the teeth scores is identified as the total tooth score, and the whole mouth mean score is calculated by averaging the total teeth scores for each animal. The plaque scoring method gives a number of 0-4 by the percentage of plaque coverage with 0=no plaque detected, 1=1-24% coverage, 2=25-49% coverage, 3=50-74% coverage and 4=75-100% coverage. Plaque thickness is given a score of 1-3 with 1=pink to light red thickness, 2=medium to red thickness and 3=dark red. The total range for the score is 0-24, e.g., each gingival and occlusal halves=plaque coverage*plaque thickness, and then each half is added together. The whole mouth score is calculated by adding the teeth scores and dividing by the number of teeth scored.

Calculus is measured by air-drying the teeth to be scored, dividing those teeth vertically into mesial, buccal, and distal thirds, and assigning each third a numerical score based on the percentage of calculus coverage with 0=no calculus detected, 1=1-24% coverage, 2=25-49% coverage, 3=50-74% coverage, and 4=75-100% coverage. Scores for each of the three tooth surfaces are added together for a tooth score and the total tooth score is divided by the number of teeth scored. The total range for the score is 0-12, e.g., each mesial, buccal and distal third=calculus coverage, and then each third is added together. The whole mouth score is calculated by adding the teeth scores and dividing by the number of teeth scored.

Dental stain is measured by dividing the teeth to be scored vertically into thirds as with the calculus index and given a numerical score for percentage of stain coverage and one for perceived stain intensity. The percentage of stain coverage is given a score of 0-4 with 0=no stain detected, 1=1-24% coverage, 2=25-49% coverage, 3=50-74% coverage and 4=75-100% coverage. The intensity is given a score of 1-3 with 1=to light yellow, tan coloring, 2=moderate brown coloring and 3=dark brown, black coloring. The score for each tooth (mesial, buccal and distal) is calculated by multiplying the coverage and intensity scores, and the resulting numbers are added to obtain a tooth score. The total range for the score is 0-36, e.g., each mesial, buccal and distal third=stain coverage*intensity, and then each third is added together. The whole mouth score is calculated by adding the teeth scores and dividing by the number of teeth measured to obtain the whole mouth score.

A total of 24 domestic cats, ranging in age from 6 years 3 months to 14 years 1 month were enrolled in this study. As noted above, on day 1 of this feeding study, the animals were provided with dental prophylaxis and on days 168 and 337, the oral substrate accumulation was assessed.

In this study, the control feline pet food composition was a commercially available feline pet food composition, Feline Control Food 1. The same feline pet food composition was supplemented with 1.2% lactic acid to provide a pet food composition designated herein as Feline Test Food 1. The data obtained are provided in Table 2:

TABLE 2

| | Group Average ± SEM | | |
|---|---|---|---|
| | Plaque | Tartar | Stain |
| Feline Control Food 1 | | | |
| 6 months | 8.1 ± 1.8 | 3.7 ± 1.2 | 3.8 ± 1.5 |
| 12 months | 7.0 ± 0.6 | 4.5 ± 0.4 | 4.8 ± 0.5 |
| Feline Test Food 1 | | | |
| 6 months | 5.6 ± 1.8 | 2.6 ± 1.2 | 2.4 ± 1.1 |
| 12 months | 4.9 ± 0.6 | 3.3 ± 0.4 | 3.2 ± 0.5 |
| Difference (%) | | | |
| 6 months | 31 | 31 | 37 |
| 12 months | 30 | 25 | 33 |

As shown by the reduction of scores for plaque, tartar and stain and the percent difference of the 6 month and 12 month scores, the data of Table 2 demonstrate that lactic acid inhibits formation of dental plaque, calculus and stain when a lactic acid containing composition comprising 1.2% lactic acid is fed to cats for six months.

### Example 2

### Lactic Acid Inhibition of Dental Plaque, Calculus, and Staining in Canines (Dogs): 28 Day Feeding Study

This study was designed to assess the effect of lactic acid on oral substrate accumulation (dental plaque, calculus, and tooth stain) in dogs. Initially, all dogs received a professional dental prophylaxis at this baseline using standard, well-established procedures. Twenty-eight days later, dental substrate accumulation (dental plaque, calculus, and tooth stain) was quantified using the Logan-Boyce quantification method as referenced and described above.

A total of 32 beagle dogs, ranging in age from 2.5 to 9 years were enrolled in this study. As noted above, on day 1 of this feeding study, the animals were provided with dental prophylaxis and on day 28, the oral substrate accumulation was assessed.

In this study, the control canine pet food composition was a commercially available canine pet food composition, Canine Control Food 1. The same canine pet food composition was supplemented with 1.5% lactic acid to provide a pet food composition designated herein as Canine Test Food 1. The analyzed amount of lactic acid for the Canine Test Food 1 was 1.1%. The Canine Control Food 1 was analyzed to have 0.2% naturally-occurring lactic acid. The data obtained are provided in Table 3:

TABLE 3

| | Group Average ± SEM | | |
|---|---|---|---|
| | Plaque | Tartar | Stain |
| Canine Control Food 1 | 7.0 ± 2.0 | 4.5 ± 2.1 | 5.3 ± 2.5 |
| Canine Test Food 1 | 6.7 ± 1.2 | 2.9 ± 1.3 | 3.2 ± 1.5 |
| Difference (%) | 4.9 | 35.7 | 38.7 |

As shown by the reduction of scores for plaque, tartar and stain and the percent difference of the control versus the test food, the data of Table 3 demonstrate that lactic acid inhibits tooth calculus and stain when a lactic acid containing dog food comprising 1.5% lactic acid is fed to dogs for one month.

### Example 3

### Lactic Acid Inhibition of Dental Plaque, Calculus, and Staining in Felines (Cats): Three Month Feeding Study

This study was designed to assess the effect of dietary lactic acid on oral substrate accumulation (dental plaque, calculus, and tooth stain), and to document evidence of bacteria. A calibration study was run prior to the study to ensure that high and low dental plaque formers were evenly distributed between the test and control groups. Initially, all cats received a professional dental prophylaxis at this calibration baseline using standard, well-established procedures. Twenty eight days later, dental substrate accumulation (dental plaque, calculus, and tooth stain) was quantified using the Logan-Boyce quantitation method as referenced and described above. Cats were then assigned to a test or control group based on dental plaque accumulation. One control group (fed a food without lactic acid supplementation) and two test groups (fed a food with lactic acid supplementation) were used, and each group had fifteen cats.

Since it was necessary to house the cats according to assigned group, a two-week acclimation period was used to allow the cats to get used to one another. Following this acclimation period, cats were given a second professional dental prophylaxis to establish a testing period baseline. After the prophylaxis, quantitative light-induced fluorescence (QLF) methods were used to capture baseline images of specific teeth according to standard procedures. The teeth assessed using the Logan-Boyce quantification method were the maxillary third incisor, canine, first, second, third, fourth premolars and first molar, and the mandibular canine, second, third, fourth premolars, and first molar. The teeth assessed using QLF were the maxillary canine, third and fourth premolars, and the mandibular canine, third and fourth premolars, and the first molar. The testing period of this study was three months, during which dental substrate accumulation and evidence of bacteria were assessed every twenty-eight days using the Logan-Boyce and QLF methods.

The control food was a feline pet food composition without added lactic acid, which was designated Feline Control Food 2. The same feline pet food composition was supplemented with 1.2% lactic acid to provide a pet food composition designated herein as Feline Test Food 2. The analyzed amount of lactic acid for the Feline Test Food 2 for Test Group 1 was 1.1% and for Test Group 2 was 1.2%. The Feline Control Food 2 was analyzed to have 0.3% naturally-occurring lactic acid. The Control Group was fed Feline Control Food 2 and Test Groups 1 and 2 were fed Feline Test Food 2. The data obtained for dental substrate accumulation for each time period are presented below.

The data obtained at the one-month time period are provided in Table 4:

TABLE 4

| | Group Average ± SEM | | |
|---|---|---|---|
| | Plaque | Tartar | Stain |
| Control Group | 5.4 ± 0.4 | 2.3 ± 0.2 | 2.1 ± 0.2 |
| Test Group 1 | 5.4 ± 0.4 | 1.9 ± 0.2 | 1.7 ± 0.2 |
| Difference (%) | 0.0 | 19.9 | 21.8 |
| Test Group 2 | 4.9 ± 0.4 | 1.8 ± 0.2 | 1.7 ± 0.2 |
| Difference (%) | 8 | 21 | 20 |

The data obtained at the two-month time period are provided in Table 5:

TABLE 5

| | Group Average ± SEM | | |
|---|---|---|---|
| | Plaque | Tartar | Stain |
| Control Group | 8.0 ± 0.5 | 3.1 ± 0.3 | 3.0 ± 0.3 |
| Test Group 1 | 7.8 ± 0.5 | 2.4 ± 0.3 | 2.3 ± 0.3 |
| Difference (%) | 2 | 23 | 21 |
| Test Group 2 | 6.5 ± 0.5 | 2.1 ± 0.3 | 2.0 ± 0.3 |
| Difference (%) | 19 | 34 | 31 |

The data obtained at the three-month time period are provided in Table 6:

TABLE 6

| | Group Average ± SEM | | |
|---|---|---|---|
| | Plaque | Tartar | Stain |
| Control Group | 8.4 ± 0.6 | 3.4 ± 0.3 | 3.4 ± 0.3 |
| Test Group 1 | 7.6 ± 0.6 | 2.8 ± 0.3 | 2.7 ± 0.3 |
| Difference (%) | 9 | 18 | 21 |
| Test Group 2 | 6.9 ± 0.4 | 2.4 ± 0.3 | 2.6 ± 0.3 |
| Difference (%) | 18 | 28 | 22 |

As shown by the reduction of scores for plaque, tartar and stain and the percent difference of the control versus the test food, the data of Tables 4-6 again demonstrate that feeding cats a pet food composition comprising elevated levels of lactic acid reduced formation of dental plaque, tartar, and stain in felines (cats). Although not presented, the QLF images qualitatively indicate that teeth of the control group cats appear to have more fluorescent substrate than the teeth of cats in either of the test groups.

What is claimed is:

1. A method of treating or reducing a dental condition in an animal, wherein the dental condition is selected from the group consisting of dental plaque, calculus, tooth staining, halitosis, stomatitis, gingivitis, periodontitis and combinations thereof, the method comprising feeding an animal in need of such treatment or reduction, an effective amount of a food composition comprising lactic acid in an amount from 0.8 weight % to about 5 weight %.

2. The method of claim 1, wherein the composition comprises lactic acid in an amount from 1.0 to 1.5 weight %, 1.0 to 1.2 weight %, or 1.1 to 1.5 weight %.

3. The method of claim 1, wherein the animal is a companion animal, optionally the companion animal is a canine or a feline.

4. The method of claim 1, wherein the dental condition is dental plaque.

5. The method of claim 1, wherein the dental condition is calculus.

6. The method of claim 1, wherein the dental condition is tooth staining.

7. The method of claim 1, wherein the dental condition is gingivitis.

8. The method of claim 1, wherein the dental condition is periodontitis.

9. The method of claim 1, wherein the composition further comprises at least one antioxidant, optionally the antioxidant is selected from the group consisting of vitamin C, vitamin E, vitamin A, lipoic acid, astaxanthin, beta-carotene, L-carnitine, coenzyme Q10, glutathione, lycopene, lutein, N-acetylcysteine, soy isoflavones, S-adenosylmethionine, taurine, tocotrienols, spinach, tomato, citrus fruit, grape, carrot, broccoli, green tea, ginkgo biloba, corn gluten meal, rice bran, algae, curcumin, marine oil, fruits, vegetables, yeast, carotenoids, flavonoids, polyphenols, and mixtures thereof.

10. The method of claim 1, wherein the animal is fed the composition for at least one month, optionally at least two months, or at least three months.

11. The method of claim 1, wherein the composition comprises one of (i) from about 10% to about 50% protein, from about 4% to about 25% fat, from about 20% to about 70% carbohydrate, and from about 4% to about 20% crude fiber, (ii) from about 30% to about 40% protein, from about 15% to about 25% fat, from about 25% to about 35% carbohydrate, and from about 5% to about 10% crude fiber, or (iii) from about 20% to about 30% protein, from about 8% to about 12% fat, from about 40% to about 60% carbohydrate, and from about 10% to about 14% crude fiber.

12. The method of claim 1, wherein the composition comprises about 1.1% lactic acid, about 33% protein, about 30% carbohydrate, about 20% fat, and about 8% crude fiber.

13. The method of claim 1, wherein the composition comprises about 1.5% lactic acid, about 25% protein, about 50% carbohydrate, about 10% fat, and about 12% crude fiber.

14. The method of claim 1, wherein the composition is a nutritionally complete companion animal pet food composition, a treat, a chew, an edible toy, or a dietary supplement.

15. The method of claim 1, wherein the composition is in the form of a coating applied to a chew or applied to an edible toy.
